(19) United States
(12) Patent Application Publication
(10) Pub. No.: US 2005/0148861 A1
(43) Pub. Date: Jul. 7, 2005

(54) Methods and systems for image selection and display

(76) Inventors: Vijay Ramanathan, Toronto (CA); Jay H. Stoffer, Culver City, CA (US)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 10/997,684

(22) Filed: Nov. 24, 2004

Related U.S. Application Data
(60) Provisional application No. 60/524,862, filed on Nov. 26, 2003.

(51) Int. Cl. A61B 5/05

(52) U.S. Cl. 600/410

(57) ABSTRACT

Methods and systems for selecting and/or displaying related images. Images may be selected by designating a first region of interest in an image, wherein the first region of interest comprises one or more addresses including an address of interest; locating one or more images in a selected series of images containing at least one address from the first region of interest; and selecting a first group of images from the selected series of images containing at least one address from the first region of interest. The selected images may be, displayed, stored, transmitted, and/or printed.

### BACKGROUND OF THE INVENTION

Digital imaging technology is essential to diagnosis of a variety of diseases including cancer, heart disease, brain disorders, and many other conditions in both humans and animals. The ability to acquire, analyze, transfer, and store detailed images of tissues and organs permits rapid and accurate diagnosis of disease and therefore better and more accurate treatment for the patient. Digital images for use in medical diagnosis are often obtained as a series of images representing adjacent slices of a target such as an organ (e.g., liver, heart, and brain) or other portion of the body. Radiologists and other medical professionals analyze the series of images to locate and identify potential pathological conditions.

Medical images may be acquired using techniques such as computed tomography (“CT”), magnetic resonance (“MR”), ultrasound, and electrical impedance tomography. CT uses x-rays to image the body. As x-rays pass through the body, they are absorbed or attenuated at various levels creating a matrix or profile of x-ray beams of different strength. A rotating frame in the CT scanner has an x-ray tube mounted on one side and a detector mounted on the opposite side. A fan-shaped beam of x-ray radiation is created as the rotating frame spins the x-ray tube and detector around the patient. Each time the x-ray tube and detector make a 360° rotation, one or more images or slices has been acquired. The images represent slices of the body, and are usually completed in a series. The spacing between slices is typically less than five millimeters. The x-ray tube and detectors are normally moved along the long axis of the body to acquire axial images that illustrate the section of anatomy being studied. Typically, one or more coronal images are constructed as scout images to indicate the positioning of each axial image. An additional series of images may be acquired after a high-contrast material is injected into the patient. A derived image series may be computed from the original series to contain different windows for display, i.e., bone, lung, brain, etc. A derived series may also be created by different computer processing techniques such as enhancement filters and/or reconstruction.

MR uses magnetic energy and radio waves to create cross-sectional images or slices of the human body. Current MR scanners perform an examination typically comprised of 2 to 12 image series. An MR series is an acquisition of data that yields a specific image orientation and a specific type of image appearance. The benefits of MR are that it can differentiate tissue and easily acquire direct views of the body in almost any orientation, while CT scanners can display fractures and typically acquire axial images. MR is typically carried out by moving a patient axially through the magnetic field of a superconducting magnet in what is called the z-direction. The patient is passed through magnetic field gradients in the horizontal x- and vertical y-direction. The excitation of nuclei in response to the magnetic field results in a series of pulses which are collected as data points representing the x, y, and z orientations. The data points are collected and used to build a spatial map showing the location of various objects in the image and assigning a unique spatial address to each object within a series of images. Often several series of images are taken of a body region (e.g., brain, heart) from different orientations (e.g., sagittal, oblique, coronal, and axial). The several series of images can be examined to give the medical professional various perspectives of the imaged target.

Medical professionals carefully examine medical images to identify and analyze pathologies or anomalies. While current medical imaging technology permits a doctor to view a pathology from various perspectives and orientations, prior imaging technology does not permit locating and displaying a particular pathology or portions of the pathology across several series of images. For example, a radiologist determines whether an apparent anomaly in one slice of one series is pathology or an artifact.

U.S. Pat. No. 6,574,629 (“the '629 patent”) refers to picture archiving and communication systems (“PACS”) for retrieving, displaying, routing, and archiving medical images. The system generally consists of an archiving station with access to long-term memory for storing image data and several reviewing stations for displaying images. According to the '629 patent, the reviewing stations can be designated to receive an image based on “routing rules” (e.g., patient location, referring physician, radiology specialty, and study status). However, the '629 patent does not permit selection or display of images based on the presence of a particular pathology, anomaly, or other location in the image.

Merge eFilm's eFilm Workstation is a diagnostic, image and manipulation tool for medical imaging. eFilm Workstation permits cross-referencing, measuring, rotation, pan, zoom, and annotation of images in addition to examination of multiple image studies simultaneously using a 3 dimensional (“3D”) cursor to select a region of interest. However, the 3D cursor is only used with the series of images currently being displayed and selects and displays one image from each series of images. Furthermore, eFilm Workstation does not eliminate series or images that do not contain a region of interest.

### BRIEF SUMMARY OF THE INVENTION

Medical imaging is a powerful tool for medical diagnosis and treatment. One or more series of images of a patient may be analyzed to identify anomalies or provide guidance for treatment of many conditions. Organization of the data is helpful to efficient and accurate analysis. Manually reading many images to locate and arrange relevant data wastes valuable resources that could be applied to other uses. Methods and systems for selecting and displaying images that are specifically relevant to analysis of an anomaly or pathology are provided that offer medical professionals a valuable tool to increase efficiency and accuracy of diagnosis and treatment.

For example, it may be advantageous for a radiologist to obtain an overview of a suspected pathology by viewing only those images containing a selected anatomic location. Images containing such anatomic location may be selected and offered for viewing. Images not containing the selected anatomic location may be eliminated, minimized, shadowed or otherwise removed from viewing. Once the radiologist has created such an overview of suspected pathology, the overview may be presented to others, such as a referring physician, to illustrate the pathology from multiple perspectives. Furthermore, simultaneous (or near-simultaneous) viewing of all pre- and post-contrast injection series aids in the determination of pathology. Similarly, comparison of the suspected pathology between current and previous studies may aid in this determination. The current study or series of images may also or alternately be compared with a reference study obtained as a normal or abnormal pathology.

Methods for selecting and displaying related images including designating a first region of interest in an image are provided. The image may be a single image or may be from at least one series comprising at least one image. The first region of interest may have a plurality of addresses and any of these may be designated as an address of interest. For ease of discussion, a single address of interest will be discussed. One or more images in a selected series of images are located and selected as containing the address of interest from the first region of interest. The one or more images form a first group of images containing the address of interest from the first region of interest. One or more images from the first group of images may be displayed.

In a further embodiment, an additional region of interest in an image may be designated. The additional region of interest may have a plurality of addresses and any of these may be designated as an additional address of interest. For ease of discussion, a single additional address of interest will be discussed. One or more images in a selected series of images are located and selected as containing the additional address of interest from the additional region of interest. The one or more images form an additional group of images containing the additional address of interest from the additional region of interest. One or more images from the additional group of images may be displayed.

Alternately, an additional address of interest in the first region of interest in an image may be designated. Thus, one or more images in a selected series of images are located and selected as containing the additional address of interest from the first region of interest. The one or more images form an additional group of images containing the additional address of interest from the first region of interest. One or more images from the additional group of images may be displayed.

Systems for selecting and displaying images are provided. Such systems comprise a computer processor; memory for storing a series of images; and a computer readable medium having a computer executable program. The computer executable program permits users to designate a first region of interest having a address of interest in an image; locate one or more images in a selected series of images containing the address of interest from the first region of interest; select a first group of images from the selected series of images having the address of interest from the first region of interest; and display at least one image from the first group of images. The computer executable program may also permit users to designate an additional region of interest having an additional address of interest in an image; locate one or more images in a selected series of images containing the additional address of interest from the additional region of interest; select an additional group of images from the selected series of images having the additional address of interest from the additional region of interest; and display at least one image from the additional group of images.

Selected images may be displayed, stored, transmitted, or printed. Storage medium (e.g., compact disc, DVD, optical disc, hard drive, memory card, RAM, and ROM) and transmissions (e.g., internet, phone, wireless, ethernet, and cable) containing images selected according to methods of the invention are provided. Images may be printed by or on any suitable medium (e.g., paper, film).

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 depicts a flowchart of a method of selecting and displaying images containing a region of interest in accordance with one embodiment of the present invention.

FIG. 2 depicts three images of a pyramid through each of five series (two axial, one coronal, and two oblique).

FIG. 3 depicts the images of FIG. 2 containing a region of interest selected and displayed from the five series of images in accordance with one embodiment of the present invention.

FIG. 4 depicts a block diagram of a system for selecting and displaying images containing a region of interest in accordance with one embodiment of the present invention.

### DETAILED DESCRIPTION OF THE INVENTION

Reference will now be made in detail to embodiments of the invention, which serve to explain the principles of the invention. It is to be understood that the application of the teachings of the present invention to a specific problem or environment will be within the capabilities of one having ordinary skill in the art in light of the teachings contained herein.

Methods and systems for selecting and/or displaying images containing a region of interest are provided. These methods and systems achieve an increased efficiency and ease in locating, selecting, and/or displaying images containing a specified region of interest (e.g., anomaly, tumor, anatomical location) using at least one address, for example an address of interest, specifying the spatial location of a region of interest in an image. Thus, images across several series of images may be searched to identify, select, and display all images containing the address of interest of the region of interest.

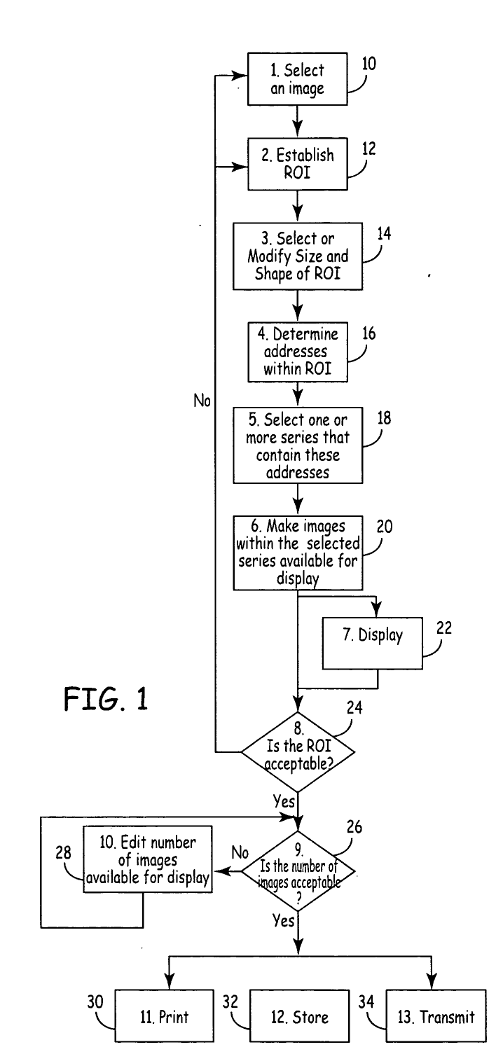

A method for selecting and/or displaying images containing a region of interest is illustrated in FIG. 1. An image is selected at block 10. The image may be a single image or may be one image in a series of images. An image may be any arrangement of addresses, locations, pixels, voxels and/or other image structure to form a spatial representation or map of an object. Images may be embodied in any medium (e.g., photographs, photocopies, transparencies, or drawings) and may be digital or analog. Images may be of any object (e.g., person, animal, machine, or device), any part of an object, or any combination of objects. Images in a series may be arranged sequentially in a given orientation (e.g., sagittal, coronal, oblique, axial) from 1 to n and represent portions or slices of an object taken as the imaging device moves across an object. In such arrangement, each slice generally represents a portion of the object of a given width (e.g., 4 mm).

A region of interest of the selected image is established at block 12. The region of interest may be established or designated in any suitable manner. Generally, a region of interest comprises at least one address representing a designated area of an object for further study. A region of interest may be designated by providing the addresses which make up the region. In one embodiment, the addresses represent the spatial boundaries of the region of interest. In another embodiment, the addresses may represent some or all of the addresses comprising the entire region of interest. The region of interest may be designated manually. For example, the region of interest may be designated by using a computer mouse to identify the addresses of a region of interest of an image displayed on a computer monitor. In such embodiment, a user clicks on or drags the mouse over or on a region of interest and thereby designates the region of interest. The size and shape of the region of interest may be selected or modified, as shown at block 14.

Addresses within the region of interest are deteremined at block 14. The addresses refer to spatial locations in an object and may be represented in any suitable manner. The address may comprise, for example, coordinates or other identifiers that represent a unique spatial location in the image. For example, an address may be represented using a coordinate system (e.g., x, y, and z). Using a coordinate system, an address may be represented in either two (x, y) or three (x, y, z) dimensions. “Pixel” generally refers to a 2D image address while “voxel” generally refers to a 3D image address. Addresses may comprise one or more pixels or voxels. Using addresses, an object may be subdivided into unique addresses forming a spatial map of an imaged object. The unique location of an address remains constant across multiple series of images of an object. For example, an address assigned to a location in an image contained in a series of sagittal images of an object also represents the same location in an image from a series of axial images of the same object.

The American College of Radiology and the National Electrical Manufacturers Association developed a standard for Digital Imaging and Communications in Medicine (“DICOM”) to faciliatate development of digital imaging communication, archiving, display, storage, and transmission. See Digitial Imaging and Communications in Medicine (DICOM) standards published by the National Electrical Manufacturers Association, Rosslyn, Va. (2003), hereby incorporated by reference in its entirety. The DICOM standard divides an image into “data elements” which is defined as a unit of information representing a single entry in the data dictionary. Id. The “data dictionary” is a registry of data elements which assign a unique tag, a name, value characteristics, and semantics to each data element. Id. A spatial map of an image can be generated in accordance with the DICOM standard, for example, to provide unique locations for data elements of an object by assigning coordinates (e.g., x, y, and z) and other identifiers to the data element.

Thus, as shown in blocks 10 through 16, a method of selecting images containing a region of interest comprises selecting at least one image from at least one series of images (block 10), designating a first region of interest in the selected image (block 12) wherein the first region of interest comprises one or more addresses, and determining addresses, including at least one address of interest, within the first region of interest (block 16). The first region of interest may have a plurality of addresses and any of these may be designated as an address of interest. For ease of discussion, a single address of interest will be discussed. However, multiple addresses may be designated.

After the address of interest within the region of interest has been determined, one or more series of images are selected, for example automatically selected, each of which contain at least one image containing the address of interest, as shown at block 18. For ease of reference, the selected one or more series of images will be referred to as the selected series of images. The image selected at block 10 may be an image in a series of images. In this case, the selected series of images comprises an image series other than the image series having the selected image. The selected series of images may be any suitable series of images. For example, a patient may have multiple series of images taken of an object (e.g., brain) at one time. Any, or several, of these series of images may be designated as the selected series. Alternatively, one or more series of images may be taken at a different time than the initial series of images and any, or several, series of images from different times may be designated as the selected series. A series of images from another patient or a series of “standard” images of the object may be designated as the selected series. Any combination of these or other series of images may be designated as a selected series.

One or more images containing the address of interest from the first region of interest are located within the selected series of images. The one or more images form a first group of images containing the address of interest from the first region of interest. As shown at block 20, images from the first group of images may be made available for display. One or more images from the first group of images may be displayed, as shown at block 22. Alternately, the first group of images may be stored, archived, printed, or transmitted.

The series selected at block 18 may comprise any images suitable for comparison with the selected image of block 10. For example, as explained above, the series selected may be a series from the same patient taken at a different time, a series from a different patient, or a series from a reference study obtained as a normal or abnormal pathology. Addresses in images from different series may be correlated using techniques for registering addresses between two or more images (e.g., correlating addresses between two or more images or selected points from the images). Image registration permits comparison of two images based on a common frame of reference. For example, size, location, or other features of a tumor may be monitored from time 1 to time 2 in order to evaluate the therapeutic impact of a particular treatment regimen. Registration of an image taken at time 1 with an image taken at time 2 permits direct comparison of the images to identify any changes in the object between time 1 and time 2. Registration may be performed by a variety of methods. See generally U.S. Pat. No. 6,553,152, hereby incorporated by reference in its entirety. For example, a transformation can be computed to map related points (i.e., landmarks) in each image. The distance between an address in image 1 and a landmark can be compared to the distance between a corresponding address in image 2 and the same landmark in image 2.

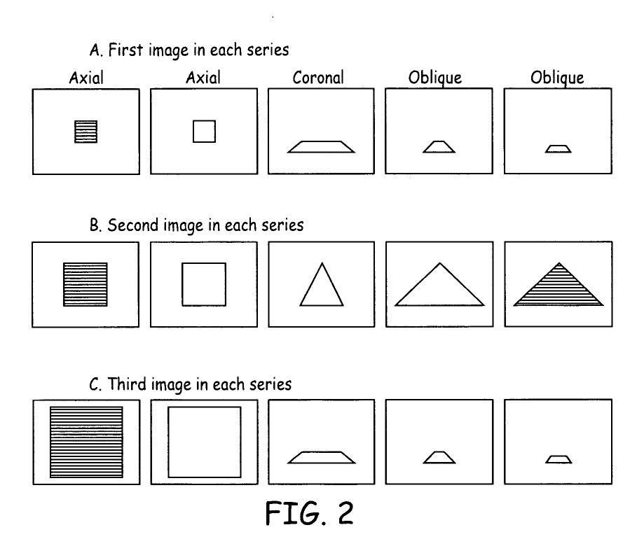

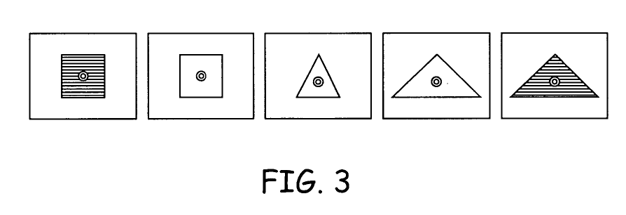

FIGS. 2 and 3 illustrate selection of images each containing an address of interest from several series of images. FIG. 2 illustrates first, second, and third images from five series of images. The five series of images comprise a pyramid: first and second axial series, a coronal series, and first and second oblique series. The first image, second image, and third image of each series is depicted in panel A, panel B, and panel C respectively. In FIG. 3, a first region of interest (represented by two concentric circles) is designated in the first axial series. As explained above, at least one address is determined within the region of interest. For ease of discussion, this is referred to as an address of interest although a pluraltiy of addresses may be selected. Images from the subsequent series containing the address of interest are located and selected to form a first group of images.

Optionally, the number of images in the first group of images may be limited to a predetermined number. For example, the number of images in the first group of images depicted in FIG. 3 may be limited to five. In another embodiment, additional images may be added to or removed from the first group of images. The additional images do not necessarily contain the address of interest from the first region of interest.

Returning to FIG. 1, after at least one image from the first group of images is displayed (block 22), a user of the system may be prompted to indicate whether the region of interest is acceptable, as shown at block 24. If the region of interest is not acceptable, the user may select a new image, shown at block 10, establish a region of interest in that image, shown at block 12, and continue until they are again prompted for whether the region of interest is accceptable, shown at block 22. If the region of interest is acceptable, the user may be prompted to indicate whether the number of images is acceptable, shown at block 26. If the number of images is not acceptable, the user may edit the number of images displayed or the number of images available for display, shown at block 28. Once the region of interest and the number of images are indicated as acceptable, the images may be printed, shown at block 30, stored, shown at block 32, or transmitted, shown at block 34.

Alternately, the user may not be prompted for whether the region of interest is acceptable and/or whether the number of images is acceptable. If not so prompted, the images may be printed, stored, or transferred, blocks 30-32, directly after being displayed at block 22.

In another embodiment, an additional region of interest may be designated in an image. The image may be the image originally selected at block 10 or may be a different image. The size or shape of the region of interest may be modified, as at block 14. At least one address is then determined within the region of interest, as at block 16. For ease of reference, this address is referred to as an additional address of interest. However, again, more than one address may be selected. As with the first region of image, after the additional address of interest within the region of interest has been determined, one or more series of images are selected including at least one image containing the additional address of interest, as at block 18. The selected series of images may be the same series of images selected in block 18 with reference to the image originally selected at block 10 or may be a different series of images. Further, a plurality of series of images may be selected. One or more images containing the additional address of interest from the additional region of interest are located within the selected series of images. The one or more images form an additional group of images containing the additional address of interest from the additional region of interest. Images from the additional group of images may be made available for display, as at block 20. One or more images from the additional group of images may be displayed, as at block 22. Alternately, the additional group of images may be stored, archived, printed, or transmitted.

Thus, for example, a user may revise or identify a different region of interest and either replace or add to the first group of images with an additional group of images containing at least one address from the additional region of interest, or otherwise maintain both groups of images.

The first and/or additional groups of images may be stored, printed, archived, discarded, or displayed on another workstation. If stored, the storage medium may be any memory location or device capable of storing images (e.g., compact disc, DVD, optical disc, hard drive, memory card, RAM or ROM).

The series of images to be assessed for presence of an image having an address of interest (the selected series of images) may be automatically generated by a computer executable program based on the region of interest. The user may manually add or remove images from the selected series of images. In one embodiment, only selected images are displayed or highlighted while images that do not contain three-dimensional points within the user's region of interest are eliminated, minimized, shadowed, or otherwise removed from view. The region of interest may be redefined by the user, triggering a reselection of images containing three-dimensional points within the new region of interest, and interactively updating the display of images. One or more regions of interest may be stored by the user for displaying, printing, transmitting, or archiving. Image registration techniques may be used to preprocess the images to align them to a constant frame of reference.

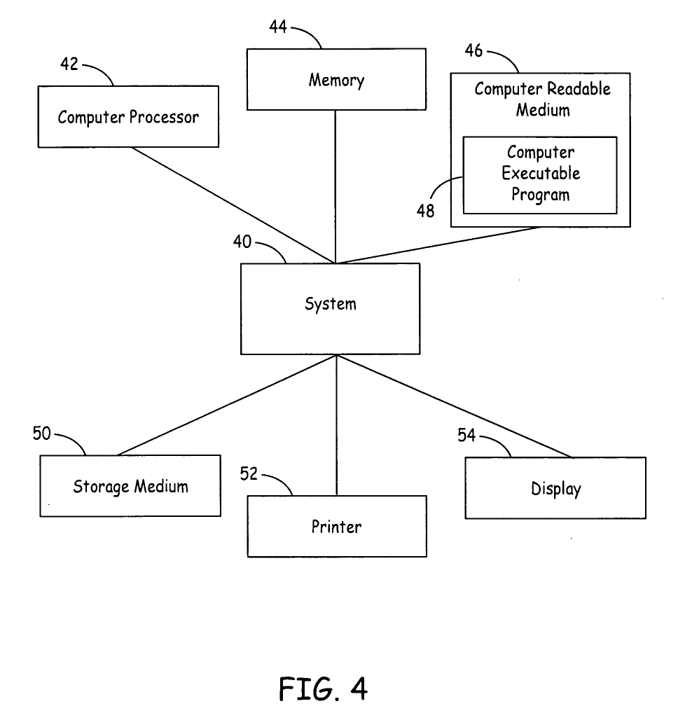

A system for displaying related images is further provided as illustrated by the block diagram of FIG. 4. The system 40 may comprise a computer processor 42, a memory for storing a series of digital images 44, and a computer readable medium 46 having a stored computer executable program 48. The stored computer executable program 48 utilizes a first region of interest having an address of interest, and locates at least one image having the address of interest in one or more selected series of one or more images. The first region of interest may include more than one address and the stored computer executable program may locate at least one image in a selected series having any address of the first region of interest. The stored computer executable program 48 may further select a first group of images from the selected series of images, wherein each of the images in the first group of images contains the address of interest (or other address from the first region of interest). The stored computer executable program 48 may display at least one image from the first group of images.

The number of images selected from the first group of images may be limited to a predetermined maximum. Optionally, images can be added or deleted from the first group of images. Images may be retrievably stored in a storage medium 50 (e.g., compact disc, optical disc, hard drive, memory card, DVD, ROM, or RAM).

In another embodiment, the computer executable program 48 may designate an additional region of interest in an image, the additional region of interest having an additional address of interest, and locate at least one image having the additional address of interest in a selected series of images. The additional region of interest may include more than one address and the stored computer executable program 48 may locate at least one image in a selected series of images having any address of the additional region of interest. The stored computer executable program 48 may further select an additional group of images from the selected series of images wherein each of the images in the additional group of images contains the additional address of interest (or other address from the additional region of interest). The stored computer executable program 48 may display at least one image from the additional group of images. Thus, for example, a user may revise or identify an additional region of interest from the first region of interest and either replace or add to the first group of images with an additional group of images containing at least one address from the additional region of interest, or otherwise retain both groups of images.

Returning to FIG. 4, the system may include means for storing, printing, archiving, discarding or displaying images on one or more workstations or servers. Thus, for example, the system may include a storage medium 50, a printer 52, and/or a display 54. The storage medium 50 may be any memory location or device capable of storing images (e.g., compact disc, DVD, optical disc, hard drive, memory card, RAM or ROM).

Storage media may be provided containing related images selected by designating a first region of interest in an image, wherein the first region of interest comprises one or more addresses including an address of interest; locating one or more images in a selected series of images containing at least one address from the first region of interest; and selecting a first group of images from the selected series of images containing at least one address from the first region of interest. The storage medium may comprise any device capable of storing images and/or any associated data (e.g., registration information, index, descriptive text, identification tags). The storage medium may be used for long or short term storage of images and may be portable (compact disc, DVD, memory card, optical disc) or relatively non-portable (e.g., storage medium installed in a mainframe computer or computer server).

Transmissions containing related images are also provided. Thus, the system may optionally include a transmitter 56 for transmitting such transmissions. The images contained in the transmission may selected by designating a first region of interest in an image, wherein the first region of interest comprises one or more addresses including an address of interest; locating one or more images in a selected series of images containing at least one address from the first region of interest; and selecting a first group of images from the selected series of images containing at least one address from the first region of interest. The transmissions may be sent over any suitable conduit (e.g., phone, satellite, cable, wireless) and/or network (internet, intranet, extranet, phone network, and wireless network).

Related images selected and/or displayed may be printed. The images to be printed may be selected by designating a first region of interest in an image, wherein the first region of interest comprises one or more addresses including an address of interest; locating one or more images in a selected series of images containing at least one address from the first region of interest; and selecting a first group of images from the selected series of images containing at least one address from the first region of interest. Selected images may be printed by or on any suitable medium (e.g., paper, film).

The above description is only illustrative of example embodiments of the present invention, and it is not intended that the present invention be limited thereto. Persons skilled in the art will recognize that changes may be made in form and detail without departing from the spirit and scope of the invention. Any modifications of the present invention which come within the spirit and scope of the following claims is considered part of the present invention.

1. A method of selecting related images comprising:
designating a first region of interest in an image, wherein the first region of interest comprises one or more addresses;
locating one or more images in a selected series of images containing at least one address from the first region of interest; and
selecting a first group of images from the selected series of images, wherein each of the images in the first group of images contains at least one address from the first region of interest.

2. The method of claim 1, wherein the number of images in the first group of images is limited to a predetermined maximum.

3. The method of claim 1, wherein a user may add or remove images from the first group of images.

4. The method of claim 1, further comprising:
designating an additional region of interest in an image, wherein the additional region of interest comprises one or more addresses;
locating one or more images in a selected series of images containing at least one address from the additional region of interest; and
selecting an additional group of images from the selected additional series of images, wherein each of the images in the additional group of images contains at least one address from the additional region of interest.

5. The method of claim 1, wherein the images are MRI images.

6. A method of displaying related images comprising:
designating a first region of interest, wherein the first region of interest comprises one or more addresses;
locating one or more images in a selected series of images containing at least one address from the first region of interest;
selecting a first group of images from the selected series of images, wherein each of the images in the first group of images contains at least one address from the first region of interest; and
displaying at least one image from the first group of images.

7. The method of claim 5, wherein the number of images selected from the first group of images is limited to a predetermined maximum.

8. The method of claim 5, wherein a user may add or remove images from the first group of images.

9. The method of claim 5, further comprising retrievably storing the first group of images in a storage medium.

10. The method of claim 9, wherein the storage medium is selected from the group consisting of compact disc, optical disc, hard drive, memory card, RAM, and ROM.

11. The method of claim 5, further comprising:
designating an additional region of interest in an image, wherein the additional region of interest comprises one or more addresses;
locating one or more images in the selected series of images containing at least one address from the additional region of interest;
selecting an additional group of images from the selected series of images, wherein each of the images in the additional group of images contains at least one address from the additional region of interest; and
displaying at least one image from the additional group of images.

12. The method of claim 11, further comprising retrievably storing the additional group of images in a storage medium.

13. The method of claim 12, wherein the storage medium is selected from the group consisting of compact disc, optical disc, DVD, hard drive, memory card, RAM, and ROM.

14. The method of claim 6, wherein the images are MRI images

15. A computer system for displaying related images, the computer system comprising:
a computer processor;
memory for storing a series of images; and
a computer readable medium having stored thereon a computer executable program comprising the steps of:
designating a first region of interest in an image, wherein the first region of interest comprises one or more addresses;
locating one or more images in a selected series of images containing at least one address from the first region of interest;
selecting a first group of images from the selected series of images, wherein each of the images in the first group of images contains at least one address from the first region of interest; and
displaying at least one image from the first group of images.

16. The system of claim 15, wherein the number of images selected from the first group of images is limited to a predetermined maximum.

17. The system of claim 15, wherein a user may add or remove images from the first group of images.

18. The system of claim 15, wherein the first group of images are retrievably stored in a storage medium.

19. The system of claim 18, wherein the storage medium is selected from the group consisting of compact disc, DVD, optical disc, hard drive, memory card, RAM, and ROM.

20. The system of claim 15, wherein the computer executable program further comprises the steps of:
designating an additional region of interest in an image, wherein the additional region of interest comprises one or more addresses;
locating one or more images in the selected series of images containing at least one address from the additional region of interest;
selecting an additional group of images from the selected series of images, wherein each of the images in the additional group of images contains at least one address from the additional region of interest; and
displaying at least one image from the additional group of images.

21. The system of claim 15, wherein the images are MRI images.

22. The system of claim 15, further comprising a storage medium for storing at least one image from the first group of images.

23. The system of claim 22, wherein the images are MRI images.

24. The system of claim 22, further comprising a transmission for transmitting at least one image from the first group of images.

25. The system of claim 24, wherein the transmission is sent over a conduit.

26. The system of claim 25, wherein the conduit is selected from the group consisting of phone, satellite, cable, and wireless.

27. The system of claim 24, wherein the transmission is sent over a network.

28. The system of claim 27, wherein the network is selected from the group consisting of internet, intranet, extranet, phone network, and wireless network.

29. The system of claim 24, wherein the images are MRI images.

30. A method of printing related images comprising:
designating a first region of interest in an image, wherein the first region of interest comprises one or more addresses;
locating one or more images in selected series of images containing at least one address from the first region of interest;
selecting a first group of images from the at least an additional series of images, wherein each of the images in the first group of images contains at least one address from the first region of interest; and
printing at least one image from the first group of images.

31. The method of claim 30, wherein the images are printed on a medium selected from the group consisting of paper and film.

32. The method of claim 30, further comprising:
designating an additional region of interest in an image, wherein the additional region of interest comprises one or more addresses;
locating one or more images in the selected series of images containing at least one address from the additional region of interest;
selecting an additional group of images from the at least an additional series of images, wherein each of the images in the additional group of images contains at least one address from the additional region of interest; and
printing at least one image from the additional group of images.

33. The method of claim 30, wherein the images are MRI images.
